(19) United States
(12) Patent Application Publication
(10) Pub. No.: US 2020/0239921 A1
(43) Pub. Date: Jul. 30, 2020

(54) CHEMOENZYMATIC SYNTHESIS OF S-NUCLEOSYL AMINO ACIDS (SNA), ANALOGS OF S-ADENOSYL-L-METHIONINE AND S-ADENOSYL-L- HOMOCYSTEINE AND USES THEREOF

(71) Applicant: ALBERT EINSTEIN COLLEGE OF MEDICINE, Bronx, NY (US)

(72) Inventors: Emmanuel Sebastien Burgos, Bronx, NY (US); David Shechter, New Rochells, NY (US)

(73) Assignee: ALBERT EINSTEIN COLLEGE OF MEDICINE, Bronx, NY (US)

(21) Appl. No.: 16/063,731

(22) PCT Filed: Jan. 25, 2017
(86) PCT No.: PCT/US17/14804

§ 371 Date: Jun. 19, 2018

Related U.S. Application Data
(60) Provisional application No. 62/290,502, filed on Feb. 3, 2016.

(51) Int. Cl. C12P 19/40; A61K 31/7064

(52) U.S. Cl. CPC C12P 19/40; A61K 31/7064

(57) ABSTRACT

Disclosed are methods for chemoenzymatic synthesis of S-Nucleosyl Amino acid probes (SNA), analogs of S-adeno-syl-L-methionine and S-adenosyl-L-homo-cysteine, analogs synthesized by the methods, and uses thereof.

### CROSS REFERENCE TO RELATED APPLICATIONS

This application claims the benefit of U.S. Provisional Application No. 62/290,502, filed on Feb. 3, 2016, the contents of which are hereby incorporated by reference.

### STATEMENT OF GOVERNMENT INTEREST

This invention was made with government support under grant number GM108646 awarded by the National Institutes of Health. The government has certain rights in the invention.

### BACKGROUND OF THE INVENTION

Protein methylation is a significant regulator of biological function and its misregulation is increasingly implicated in oncogenesis and tumor progression. Protein arginine methyltransferases (PRMTs), a family of nine enzymes in humans, methylate arginines in many proteins, including histones, spliceosomal factors, and ribosomal proteins (FIG. 1). Since PRMTs are critical components of a range of biological processes and are frequently misregulated in cancer, these enzymes are emerging targets for chemotherapy. In particular, PRMT5 is overexpressed in tumors and its elevated activity is highly correlated with poor clinical prognosis.

S-adenosyl-L-methionine (SAM) is the universal methyl donor substrate for all methyltransferases and thus a scaffold for drug design. The present invention addresses the need for analogs of SAM and S-adenosyl-L-homocysteine (SAH) for therapeutic treatments.

### SUMMARY OF THE INVENTION

The present invention provides various strategies to prepare S-Nucleosyl Amino acid probes (SNA). A ‘tool-box’ composed of recombinant promiscuous enzymes (e.g. kinases, methionine adenosyltransferases, SAM-dependent chlorinase, and/or S-adenosyl-L-homocysteine hydrolase) is introduced for the preparation of SAM-cofactor and SAH analogs with broad chemical diversity. Using an immunoassay and/or a radioactive assay, the SNA library is screened against various methyltransferases to pin-point valuable chemical scaffolds and to further assist in the synthesis of selective methyltransferases' inhibitors.

The present invention provides methods of synthesizing an analog of S-adenosyl-L-methionine (SAM) comprising: i) reacting a nucleoside or nucleoside analog with a nucleoside triphosphate in the presence of one or more of adenosine kinase, deoxynucleoside kinase and deoxycytidine kinase to form a monophosphate nucleoside analog; ii) forming a triphosphate nucleoside analog by a) reacting the monophosphate nucleoside analog with a nucleoside triphosphate in the presence of one or both of myokinase and cytidylate kinase to form a diphosphate nucleoside analog; and reacting the diphosphate nucleoside analog with a nucleoside triphosphate in the presence of one or both of pyruvate kinase and nucleoside-diphosphate kinase to form a triphosphate nucleoside analog; and/or b) reacting the monophosphate nucleoside analog with phosphoenolpyruvic acid and pyrophosphate in the presence of pyrophosphate phosphate dikinase to form a triphosphate nucleoside analog; and iii) reacting the triphosphate nucleoside analog with methionine or a methionine analog in the presence of methionine adenosyltransferase to form an analog of SAM.

Also provided are methods of synthesizing an analog of S-adenosyl-L-methionine (SAM) comprising reacting a 5′-chlorinated nucleoside analog with methionine or a methionine analog in the presence of a SAM-dependent chlorinase to form an analog of SAM.

Also provided are methods of synthesizing an analog of S-adenosyl-L-homocysteine (SAH) through reaction between a nucleoside or nucleoside analog with homocysteine or an analog of homocysteine (e.g. cysteine) in the presence of S-adenosyl-L-homocysteine hydrolase to form an analog of SAH.

Also provided are disclosed analogs and their use in treating cancer.

### BRIEF DESCRIPTION OF THE DRAWINGS

FIG. 1. All protein arginine methyltransferases (PRMTs) catalyze methylation of arginine residues to generate monomethyl arginine (MMA). While the PRMT1, PRMT3, PRMT4, PRMT6 and PRMT8 catalyze the formation of asymmetric dimethyl arginine (aDMA), the symmetric dimethyl arginine mark (sDMA) is deposited by the PRMT isozymes 5, 7 and 9.

FIG. 2. SAM substrate and SAH analog interaction with the human methyltransferases Dot1L, CARM1 (i.e. PRMT4) and PRMT1. Overview of Dot1L structure (ribbon and surface representation) with close-ups on the cofactor binding pocket (SAM-bound in first frame, PDB: 3QOW, and N₆-methyl SAH inhibitor-bound in second frame, PDB: 3SR4; black sticks). Table of inhibition constants (Kᵢ; μM) for several molecules (1, 2 and 3, depicted on far-right) against human Dot1L, CARM1 and PRMT1.

FIG. 3A-3C. An overview of the SAM binding pocket from three members of the PRMT family. Ribbon and surface shown, far left. Close-ups of the cofactor binding pocket depict the ‘adenosine’ and the ‘methionine’ binding mode (left and right, respectively). Black stick representation show SAM or its bound analogs. A. human PRMT5 (PDB: 4GQB; HsPRMT5). B. rat PRMT1 (PDB: 1ORH; RnPRMT1). C. human PRMT4 (PDB: 4IKP; HsCARM1). Dashed lines=hydrogen bonds; structural water molecules are depicted with spheres.

FIG. 4A-4C. Chemoenzymatic approach to the synthesis of a SAM-cofactor library is more efficient than chemical synthesis. A. Comparison between the tedious lengthy chemical approach (shaded) and the two chemoenzymatic roads leading to the correct SAM stereoisomer. Strategies first employ a nucleoside activation (left arrow) followed by a nucleophilic displacement (right arrow). The activation steps through phosphorylation are catalyzed by enzymes (oval shapes): adenosine kinase (AK), myokinase (MK) and pyruvate kinase (PK). Then, nucleophilic displacement with methionine is catalyzed either by SAM-synthetases (MAT) on the nucleoside triphosphates (top approach) or by SAM-dependent chlorinase (SalL) on the 5′-chloro-5′-deoxy adenosine (bottom approach). B. Coomassie-stained SDS-PAGE showing, from left to right, recombinant enzymes from Salinispora tropica (StSalL), Methanococcus jannaschii (MjMAT), Sulfolobus solfataricus (SMAT), Neisseria meningitidis (NmMAT) and Campylobacter jejuni (CjMAT). C. Sampling of the chemical space around the SAM methyl donor includes alterations onto the nucleobase (“BASE”), the sugar moiety (“SUGAR”) and the amino-acid tail (“METHIONINE”). Modifications are represented by atoms (where X, Y and Z are either a nitrogen, a carbon, a sulfur, or an oxygen atom) and functional groups where R₁₋₁₀ are either a hydrogen-, an oxygen-, a fluorine-, a chlorine-, or a sulfur-atom, a hydroxyl-, an ether-, an ester-, a carboxylic acid-, a cyano-, an azido-, a primary- secondary- or tertiary-amino-, or a hydrocarbon-group (e.g. methyl, ethyl).

FIG. 5A-5C. Approaches to the preparation of SAM and SAH analogs. A. Enzyme ‘tool box’ for the synthesis of nucleoside triphosphates. Enzymes (oval shapes) used include: deoxynucleoside kinase (dNK), deoxycytidine kinase (dCK), cytidylate kinases (dCMPK, CMPK), nucleoside-diphosphate kinase (NDK) and pyrophosphate phosphate dikinase (PPDK). B. Lupinus luteus adenosylhomocysteinase (L/SAHH) assisted synthesis of SAH analogs. S-adensoyl-L-cysteine is synthesized through the L/SAHH-catalyzed reaction using adenosine (ADO) and cysteine (n=1). C. Chemical synthesis of 5′-triphosphate adenosines (ADO).

FIG. 6. An overview of the methionine analog specificity for recombinant SAM-synthetases (MAT) and SAM-dependent chlorinase from Salinispora tropica (StSalL). The relative enzymatic efficiencies of four molecules (M1-4) were measured. These values are compared for the enzymes from M. jannaschii (MjMAT; black), N. meningitidis (NmMAT; stripped pattern), C. jejuni (CjMAT; grey) and S. tropica (StSalL; squared pattern).

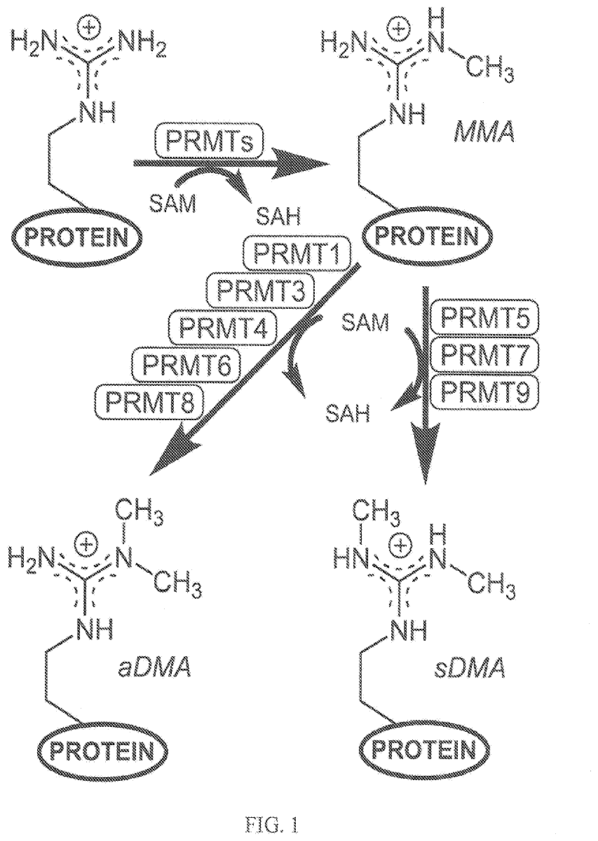

### DETAILED DESCRIPTION OF THE INVENTION

The invention provides various strategies to prepare S-Nucleosyl Amino acid probes (SNA). A ‘tool-box’ composed of recombinant promiscuous enzymes (e.g. kinases, methionine adenosyltransferases, SAM-dependent chlorinase, and/or S-adenosyl-L-homocysteine hydrolase) is introduced for the preparation of SAM-cofactor and SAH analogs with broad chemical diversity. Using an immunoassay and/or a radioactive assay, the SNA library is screened against various methyltransferases to pin-point valuable chemical scaffolds and to further assist in the synthesis of selective methyltransferases' inhibitors.

The invention provides a method of synthesizing an analog of S-adenosyl-L-methionine (SAM) comprising

i) reacting a nucleoside or nucleoside analog with a nucleoside triphosphate in the presence of one or more of adenosine kinase, deoxynucleoside kinase and deoxycytidine kinase to form a monophosphate nucleoside analog;

ii) forming a triphosphate nucleoside analog by
a) reacting the monophosphate nucleoside analog with a nucleoside triphosphate in the presence of one or both of myokinase and cytidylate kinase to form a diphosphate nucleoside analog; and reacting the diphosphate nucleoside analog with a nucleoside triphosphate in the presence of one or both of pyruvate kinase and nucleoside-diphosphate kinase to form a triphosphate nucleoside analog; and/or
b) reacting the monophosphate nucleoside analog with phosphoenolpyruvic acid and pyrophosphate in the presence of pyrophosphate phosphate dikinase to form a triphosphate nucleoside analog; and

iii) reacting the triphosphate nucleoside analog with methionine or a methionine analog in the presence of methionine adenosyltransferase to form an analog of SAM.

Any of the nucleoside triphosphates can be, for example, adenosine triphosphate (ATP) or cytidine triphosphate (CTP).

The adenosine kinase can be, for example, Anopheles gambiae adenosine kinase. The deoxynucleoside kinase can be, for example, Bacillus anthraces deoxynucleoside kinase. The deoxycytidine kinase can be, for example, Homo sapiens deoxycytidine kinase. The cytidylate kinase can be, for example, Coxiella burnetii cytidylate kinase. The nucleoside-diphosphate kinase can be, for example, Coxiella burnetii nucleoside-diphosphate kinase. The pyrophosphate phosphate dikinase can be, for example, from Zea mays or Clostridium symbiosum. The methionine adenosyltransferase can be, for example, from Methanococcus jannaschii, Sulfolobus solfataricus, Neisseria meningitidis or Campylobacter jejuni.

Also provided is a method of synthesizing an analog of S-adenosyl-L-methionine (SAM) comprising reacting a 5′-chlorinated nucleoside analog with methionine or a methionine analog in the presence of a SAM-dependent chlorinase to form an analog of SAM. The SAM-dependent chlorinase can be, for example, Salinispora tropica enzyme.

Also provided are methods of synthesizing an analog of S-adenosyl-L-methionine (SAH) comprising reacting a nucleoside or nucleoside analog with homocysteine or an analog of homocysteine (e.g. cysteine) in the presence of S-adenosyl-L-homocysteine hydrolase to form an analog of SAH. The S-adenosyl-L-homocysteine hydrolase can be, for example, Lupinus luteus enzyme.

The nucleoside or nucleoside analog can be a ribonucleoside or ribonucleoside analog or a 2′-deoxyribonucleoside or 2′-deoxyribonucleoside analog or a 3′-deoxyribonucleoside or 3′-deoxyribonucleoside analog. The nucleoside or nucleoside analog can be, for example, adenosine, 2′-deoxyadenosine, 3′-deoxyadenosine, an analog of adenosine, an analog of 2′-deoxyadenosine, or an analog of 3′-deoxyadenosine. The nucleoside analog can be substituted, for example, with one or more of methyl, ethyl, benzyl, —NH₂, —OH, —SH, —NHNH₂, —NHOH, —NO₂, —N₃, —F, —Cl, ═O and —CD₃. Examples of nucleoside analog that can be used include, but are not limited to, tubercidin, vidarabine, 2-amino adenosine, 2-fluoro adenosine, 2′-fluoro-2′-deoxy adenosine, 2′-amino-2′-deoxy adenosine and N₆-methyl adenosine.

One or more of adenosine kinase, deoxynucleoside kinase, deoxycytidine kinase, myokinase, cytidylate kinase, pyruvate kinase, nucleoside-diphosphate kinase, pyrophosphate phosphate dikinase, methionine adenosyltransferase, SAM-dependent chlorinase and S-adenosyl-L-homocysteine hydrolase used in the methods disclosed herein can be a recombinant enzyme expressed, for example, in E. coli.

SAM analogs that can be used, include for example, the following:

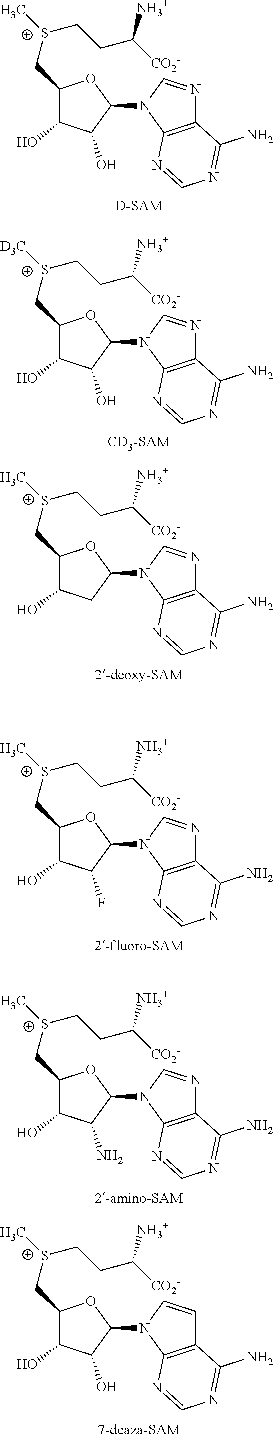

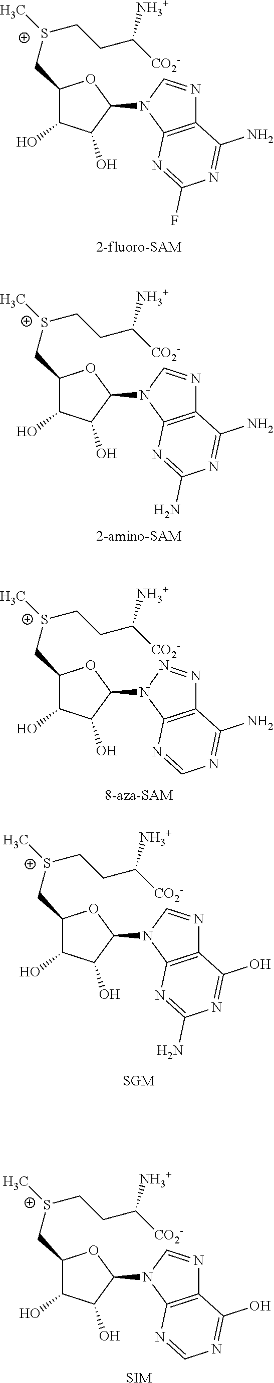

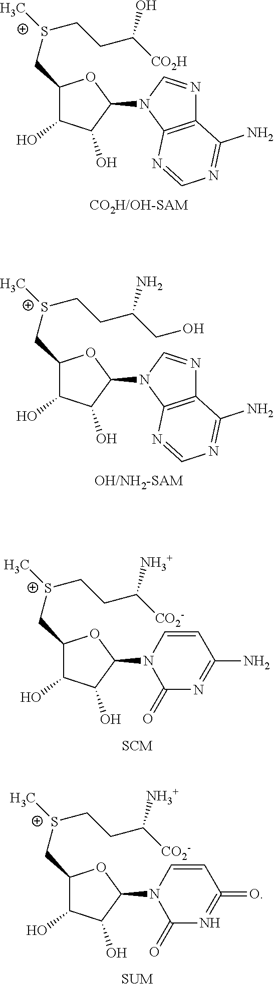

SAH analogs that can be used, include for example, the following:

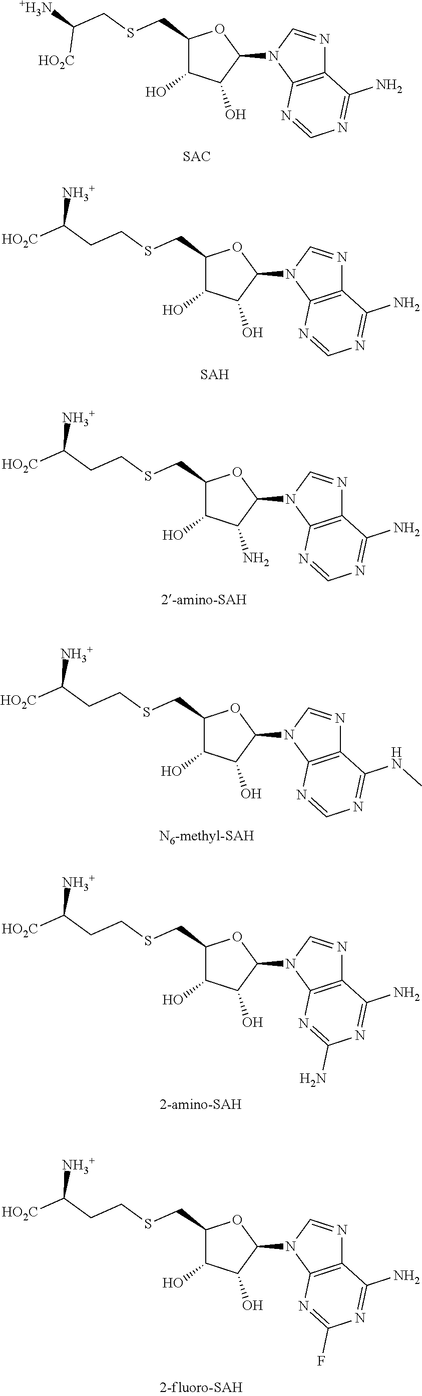 [SMILES: Nc1ncnc2c1ncn2[C@@H]1O[C@H](CSC[C@H]([NH3+])C(=O)O)[C@@H](O)[C@H]1O ; Nc1ncnc2c1ncn2[C@@H]1O[C@H](CSCC[C@H]([NH3+])C(=O)O)[C@@H](O)[C@H]1O ; Nc1ncnc2c1ncn2[C@@H]1O[C@H](CSCC[C@H]([NH3+])C(=O)O)[C@@H](O)[C@H]1N ; CNc1ncnc2c1ncn2[C@@H]1O[C@H](CSCC[C@H]([NH3+])C(=O)O)[C@@H](O)[C@H]1O ; Nc1nc(N)c2ncn([C@@H]3O[C@H](CSCC[C@H]([NH3+])C(=O)O)[C@@H](O)[C@H]3O)c2n1 ; Nc1nc(F)nc2c1ncn2[C@@H]1O[C@H](CSCC[C@H]([NH3+])C(=O)O)[C@@H](O)[C@H]1O]

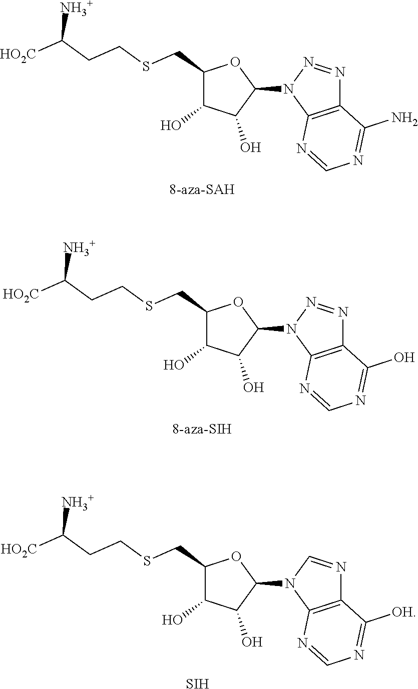 [SMILES: Nc1ncnc2c1nnn2[C@@H]1O[C@H](CSCC[C@H]([NH3+])C(=O)O)[C@@H](O)[C@H]1O ; [NH3+][C@@H](CCSC[C@H]1O[C@@H](n2nnc3c(O)ncnc32)[C@H](O)[C@@H]1O)C(=O)O ; [NH3+][C@@H](CCSC[C@H]1O[C@@H](n2cnc3c(O)ncnc32)[C@H](O)[C@@H]1O)C(=O)O ; "SIH"]

The invention also provides a compound selected from the group consisting of

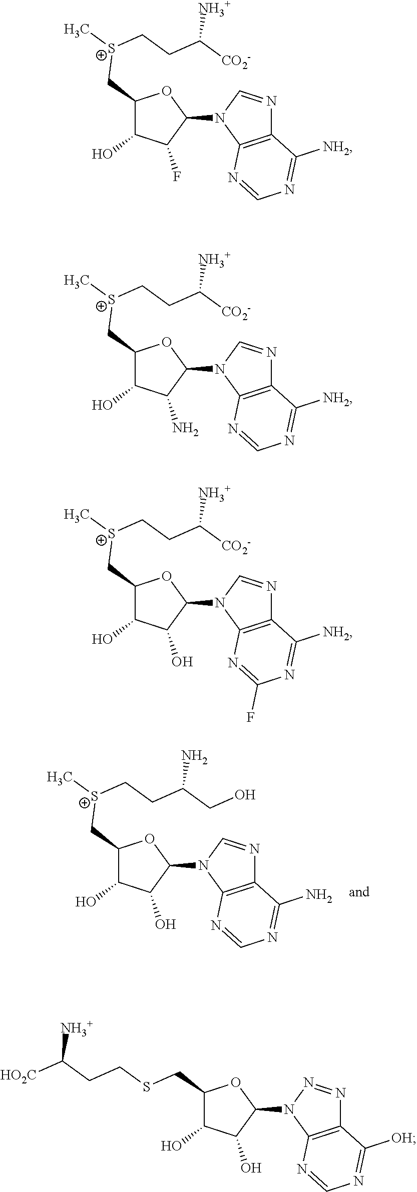

or a pharmaceutically acceptable salt thereof.

Pharmaceutically acceptable salts that can be used with compounds of the present invention include, e.g., non-toxic salts derived, for example, from inorganic or organic acids including, but not limited to, salts derived from hydrochloric, sulfuric, phosphoric, acetic, lactic, fumaric, succinic, tartaric, gluconic, citric, methanesulphonic and p-toluenesulphonic acids.

Also provided is a pharmaceutical composition comprising one or more of the compounds disclosed herein and a pharmaceutically acceptable carrier. Pharmaceutically acceptable carriers and diluents that can be used herewith encompasses any of the standard pharmaceutical carriers or diluents, such as, for example, a sterile isotonic saline, phosphate buffered saline solution, water, and emulsions, such as an oil/water or water/oil emulsions. The pharmaceutical compositions can be formulated to be advantageous for the selected route of administration to a subject.

Also provided is a method of treating a cancer in a subject comprising administering to the subject one or more of the compounds disclosed herein in an amount effective to treat a cancer in a subject. The compound can be administered in an amount effective to inhibit a methyltransferase involved in cancer development or progression in a subject.

As used herein, “treating” or “treat” a cancer means to alleviate or ameliorate or eliminate a sign or symptom of the cancer that is being treated. For example, treatment with the compound can reduce or eliminate the cancer in the subject, or retard the growth, development, progression or spread of the cancer in the subject.

The compounds and compositions of the present invention can be administered to subjects using routes of administration known in the art. The administration can be systemic or localized to a specific site. Routes of administration include, but are not limited to, intravenous, intramuscular, intrathecal or subcutaneous injection, oral or rectal administration, and injection into a specific site.

All combinations of the various elements described herein are within the scope of the invention unless otherwise indicated herein or otherwise clearly contradicted by context.

This invention will be better understood from the Experimental Details, which follow. However, one skilled in the art will readily appreciate that the specific methods and results discussed are merely illustrative of the invention as described more fully in the claims that follow thereafter.

### EXPERIMENTAL DETAILS

### Introduction

All methyltransferases use the universal S-adenosyl-L-methionine (SAM) methyl-donor. Recent computational studies have demonstrated the druggability of the SAM binding pocket for methyltransferases, including PRMTs. Disclosed herein is a chemoenzymatic approach for production of SAM/SAH analogs with broad chemical diversity. These molecules are further referred to as S-Nucleosyl Amino acids or SNA. As a proof of concept, starting from commercial nucleoside, methionine and homocysteine analogs, fourteen SNA were synthesized through simple use of recombinant enzymes expressed in E. coli. In vivo, methionine adenosyltransferase (MAT) catalyzes the formation of SAM from L-methionine and adenosine triphosphate (ATP). The present approach uses an array of recombinant enzymes, including MAT proteins with broad substrate specificity. This unique combination of enzymes is the key to a fast and stereoselective ‘one-pot’ preparation of SAM/SAH analogs. This method will promote a completely new approach to the development of small molecules to target methyltransferases, including PRMT-specific inhibitors.

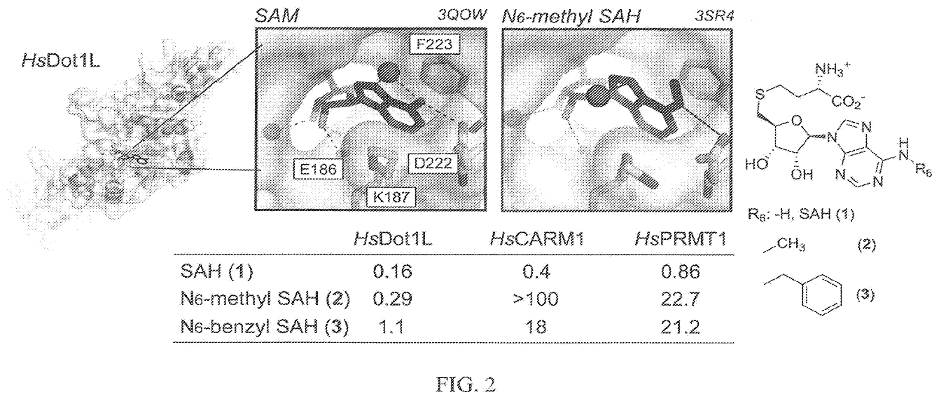

Several attempts have been made to identify inhibitors selective over different human PRMTs, yet many were not truly isozyme-specific (for example, selectivity for PRMT5/6 over CARM1 below 30). Although PRMTs have a broad range of methyl acceptors, the arginine substrate is often isozyme-specific so novel bi-substrate scaffolds occupying the arginine and SAM binding sites were synthesized. Yet these inhibitors display low potency and poor selectivity. Instead, the properties described are reminiscent of the naturally occurring inhibitor S-adenosyl-L-homocysteine by-product of all methyl-transferase reactions (SAH; FIG. 2, molecule 1).

To advance the design and utility of inhibition of arginine methyltransferases for cancer chemotherapy, scaffolds of SAM or SAH analogs are expected to be effective and selective inhibitors of PRMTs. Recent computational analyses based on structural biology studies predict that the SAM cofactor binding site is chemically tractable. Phylogenetic sequence alignment of methyltransferase active sites predicts high similarity between: 1) PRMTs, 2) small molecule methyltransferases (SMMTs) and 3) the disrupter of telomere silencing 1-like enzyme (Dot1L). SAH is a potent, yet non-specific, inhibitor of methyltransferases (FIG. 2; molecule 1, first row from table) and the genetic similarities mentioned above support this observation. SAM and ATP share similarities (i.e. adenosyl group) and so do their binding pockets. Nucleoside analogs have highly proven effectiveness in the clinic as there are various examples of selective and specific kinases inhibitors, supporting the approach for SAM analogs.

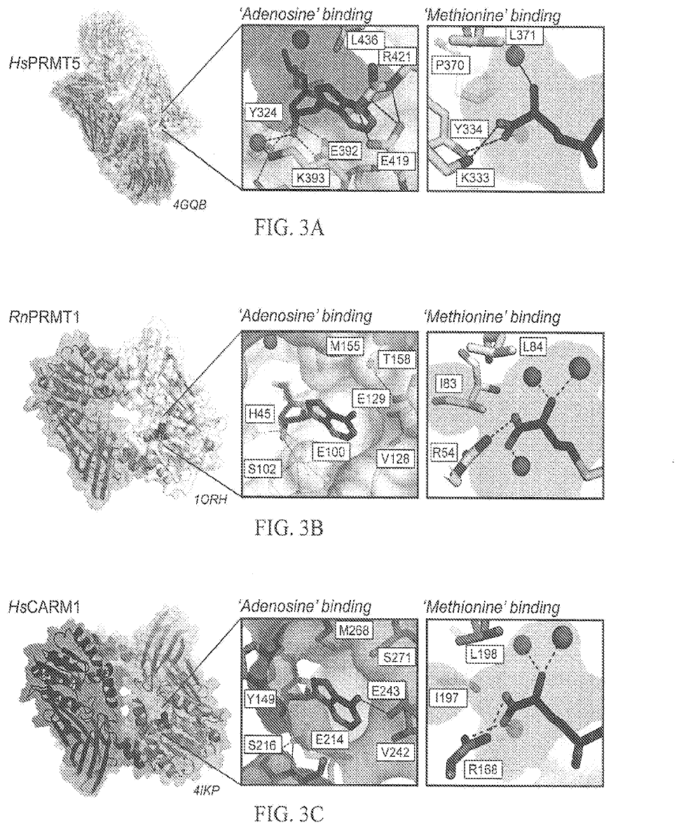

In support of this hypothesis, SAM or SAH analogs harboring subtle chemical modifications display potent affinity toward distinct methyltransferases. S-(5′-adenosyl)-3-thiopropylamine (decarboxylated analog of SAH) is 10-fold more selective for PRMT1 over PRMT6. The N₆-methyl SAH molecule (FIG. 2, molecule 2) perfectly illustrates how selectivity can emerge from a slight alteration of the SAM/SAH original scaffold as it is 350- and 80-fold selective for Dot1L over CARM1 and PRMT1, respectively (FIG. 2; second row in table). Several cofactor PRMT binding pocket differences emerge upon structural analysis, further supporting the hypothesis (FIG. 3). The occupancy for the nucleoside binding site in PRMT5 (FIG. 3A; first close-up) diverges from the highly exposed ‘adenosine’ moiety in PRMT1 (FIG. 3B; first close-up) and the moderate disposition for CARM1 to interact with solvent (FIG. 3C; first close-up). The L-homocysteine binding site is often filled with structural water molecules (FIG. 3; far-right close-ups). Thus, chemical alterations to enhance the hydrogen bond network in this portion of the active site may lead to the discovery of unnatural cofactor substrates with unprecedented isozyme specificity.

### Experimental Procedures

### 1.1 Production of Nucleoside Triphosphate Building Blocks

1.1.a ‘One-Pot’ Chemoenzymatic Synthesis of Nucleoside Triphosphates to Access SAM Analogs.

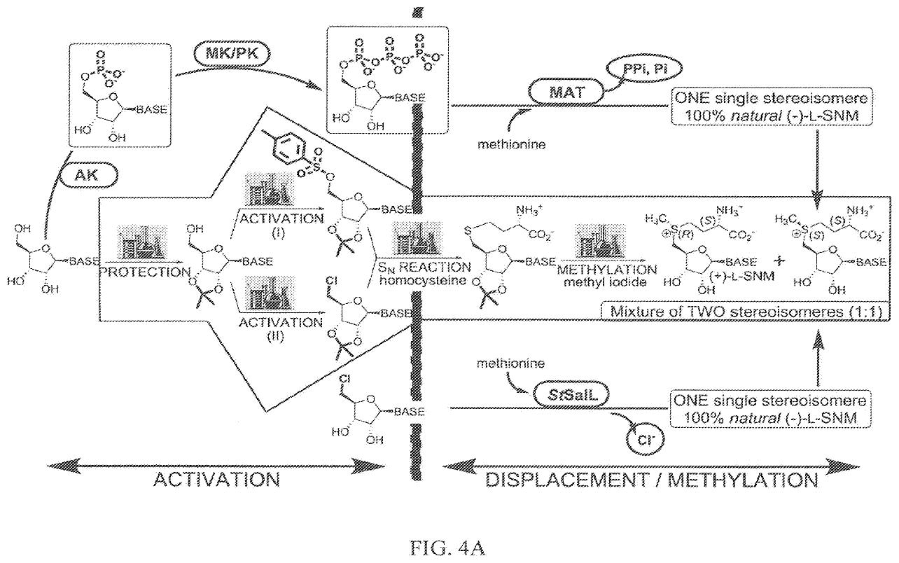

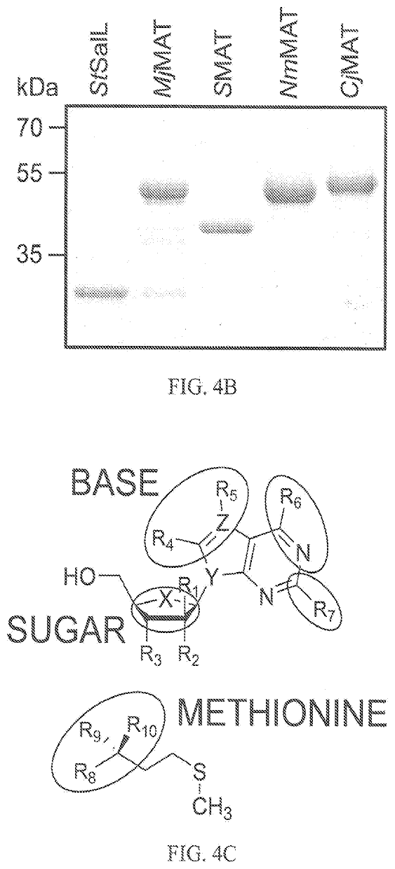

Synthetic chemical approaches yield a 1:1 mixture of the two SAM stereoisomers (FIG. 4A, shaded middle frame). The enzymes that catalyze the formation of SAM from ATP and L-methionine in vivo are used to produce SAM analogs (MAT; FIG. 4A, top enzymatic approach). Adenosine phosphorylation is performed by kinases (e.g. adenosine kinase ‘AK’, myokinase ‘MK’ and pyruvate kinase ‘PK’; FIG. 4, top enzymatic approach). MAT proteins are well characterized and several mechanistic and structural biology reports make these enzymes the preferred biocatalyst for rapid access to SAM analogs. Methanococcus jannaschii MAT has been purified (MjMAT; FIG. 4B). The promiscuity of this SAM-synthetase and its ability to utilize an array of nucleoside triphosphates other than ATP (e.g. dATP, GTP, ITP, CTP, UTP) was confirmed using high-performance liquid chromatography (HPLC).

Library Starting Material.

Customized libraries of adenosine analogs can be acquired from commercial sources (i.e. Carbosynth and Granlen). These molecules will cover chemical alterations on the ‘sugar’ (FIG. 4C; R₁, R₂, R₃ and X) and the ‘base’ (FIG. 4C; R₄, R₅, R₆, R₇, Y and Z). Each single point variation will either increase hindrance (e.g. N- or O-methyl/ethyl substitutions) or modulate hydrogen bonding capacity (e.g. —NH₂, —OH, —SH, —NHNH₂, —NHOH, —NO₂, —N₃, —F, —Cl, —CH₃) of the final compound.

Synthesis of Nucleoside Triphosphate Analogs.

To produce nucleoside triphosphate analogs, AK, MK and PK enzymes are used to catalyze the formation of nucleoside mono-, di- and triphosphate analogs, respectively (FIG. 4A; top enzymatic approach). The MK and PK enzymes are commercially available (Sigma), and AK from Anopheles gambiae (AgAK) was expressed and purified in E. coli. Importantly, AgAK displayed good catalytic efficiency (k_{cat}/Kₘ˜1.0×10⁴ M⁻¹·s⁻¹) for several adenosine analogs tested (e.g. tubercidin, vidarabine, 2-amino adenosine, 2-fluoro adenosine, 2′-fluoro-2′-deoxy adenosine, 2′-amino-2′-deoxy adenosine, N₆-methyl adenosine) showing its utility for the screen in milligram quantities with >95% yields within 3 hours. Upon removal of the enzymes by ultrafiltration, the nucleoside triphosphate (NTP) can be purified by reverse-phase HPLC. Following optimization, preparations can be scaled-up (25 mg nucleoside; 1 mL reaction) and purification carried by semi-preparative reverse-phase HPLC. A last desalting step (i.e. HPLC) can be performed to provide salt-free NTPs. After concentration by freeze-drying, NTPs can be stored in −80° C. at a 100 mM stock concentration. These NTPs are enzymatically combined with methionine analogs to produce SNMs.

1.1.b Overcoming Poor Enzymatic Conversions.

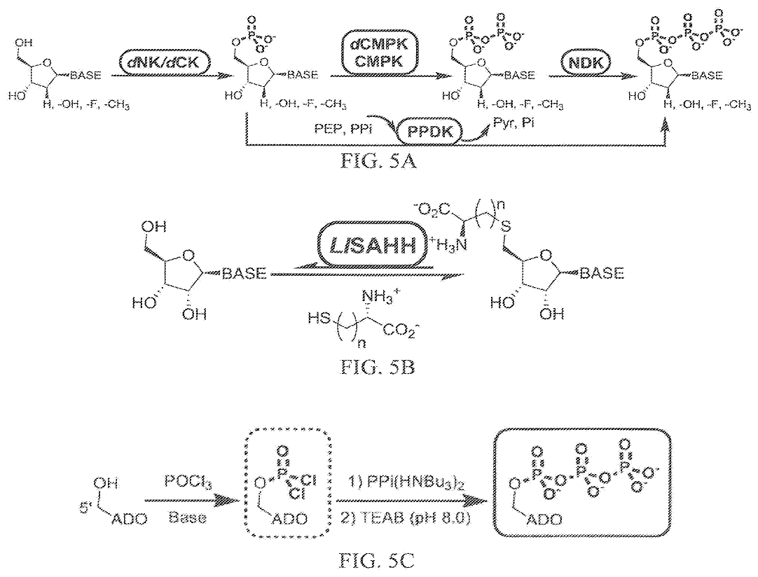

Although the AK/MK/PK enzymatic system is promiscuous, a few analogs are resilient to this approach. Molecules bearing an arabinofuranosyl configuration (FIG. 5A; e.g. 942′-deoxy-2′-fluoro-β-D-arabinofuranosylladenine) displayed low reactivity so complementary enzymes were prepared. The preparation of these enzymes was straightforward since two of them were already purified by the New York Structural Biology Center at Einstein (NYSBC): deoxynucleoside kinase (dNK; from Bacillus anthraces, DNASU clone ID FLH249881.01F), deoxycytidine kinase (dCK; from Homo sapiens, DNASU clone ID FLH271744.01L), cytidylate kinases (dCMPK, CMPK; from Coxiella burnetii, NYSBC clone ID CBU_0527), nucleoside-diphosphate kinase (NDK; from Coxiella burnetii, NYSBC clone ID CBU_1258). Another two enzymes, the pyrophosphate phosphate dikinases (PPDK) from Zea mays and Clostridium symbiosum, are also useful (FIG. 5B). These PPDK proteins catalyze the conversion from mono- to nucleoside triphosphate using PEP and pyrophosphate (PPi). Finally, a chemical approach may be used to synthesize nucleoside triphosphates (FIG. 5C).

1.2 SMN Assembly from NTPs and Methionine Analogs

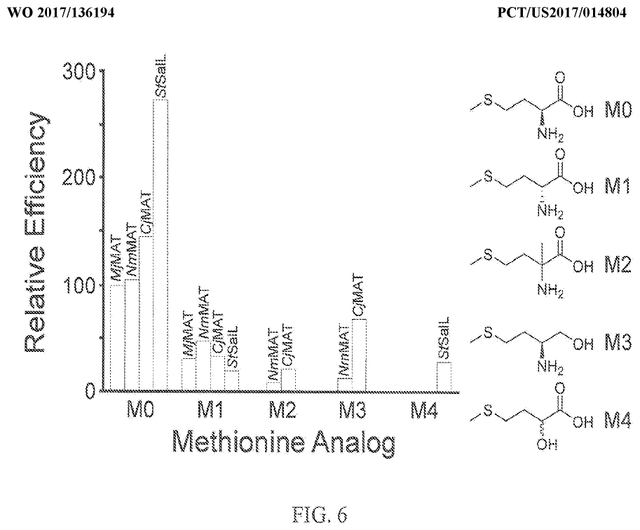

An enzyme array to incorporate a broad spectrum of methionine analogs. To expand the SNA library, the reactivity of several methionine analogs (FIG. 4C, amino-acid tail labelled ‘METHIONINE’) was screened toward MjMAT in the presence of ATP. Since only L- and D-methionine (M0 and M1, respectively; FIG. 6) were efficiently processed by MjMAT, other species' MAT enzymes originally described for synthesis of sulfonium-alkyl SAM-analogs and ester derivatives were screened. MAT enzymes from Neisseria meningitidis (Nm) and Campylobacter jejuni (Cj) (FIG. 4B) were expressed and purified. To demonstrate the utility of these enzymes, α-methyl-methionine (M2, FIG. 6) was a substrate of Nm- and CjMAT (FIG. 6) and (S)-(−)-methioniol (M3; FIG. 6) was an excellent substrate for CjMAT.

Since reactivity was not observed toward analog M4 for any MAT enzyme, a complementary approach to the chemoenzymatic synthesis of SNA probes was used (FIG. 4A, bottom enzymatic road). The unique SAM-dependent chlorinase from the marine organism Salinispora tropica (StSalL) is a key enzyme for salinosporamide A biosynthesis. Although the occurrence of high chloride concentration (i.e. 546 mM) in sea water shifts the chemical equilibrium away from SAM synthesis, this reaction is reversible in vitro. StSalL (FIG. 4B) was expressed and purified, and the methionine analog specificity for this enzyme was determined in the presence of 5′-chloro-5′-deoxy adenosine (5′-ClDA; FIG. 6). Reactivity was observed toward 2-hydroxy-4-(methylthio) butanoic acid (M4; FIG. 6).

### 1.3 Combine the Above Tools to Produce a SAM Analog Library

Nucleophilic displacements by methionines to yield S-Nucleosyl Amino acid probes (SNA). Small scale reactions for each catalyst were performed using the NTP, methionine analogs and HPLC method. The best enzyme for the synthesis of each SNM is determined including the determination of corresponding kinetic parameters (FIG. 4A, production of PPi from MAT reaction) or by HPLC. Finally, the SNM analogs are produced on a larger scale, with purification by HPLC for subsequent screening. Molecules are characterized through nuclear magnetic resonance (NMR). Chemical identity is also confirmed by mass spectrometry.

Alternative chemoenzymatic routes. A few SNA probes are difficult to prepare. To broaden the substrate diversity, the reversible reaction catalyzed by S-adenosyl-L-homocysteine hydrolase from Lupinus luteus (L/SAHH) that is displaced toward SAH production in vitro was used (FIG. 5B). The enzyme had a poor nucleoside specificity and could utilize both L-homocysteine and L-cysteine (n=2 and n=1, respectively; FIG. 5B). This powerful catalyst was a useful tool for production of S-nucleosyl-L-homocysteine and S-nucleosyl-L-cysteine analogs (SNH and SNC, respectively). Unlike SAM analogs, SNH and SNC probes are not substrates for PRMTs, so they are assayed as competitive inhibitors.

### 2.1 Preparation of Arginine Substrate Acceptors for PRMT Screens

SAM analogs will be assayed toward each methyltransferases and PRMT isozymes using specific arginine substrates. Nucleoplasmin has been used to test profiling of HsPRMT5-MEP50. Histone H4 is also used as a substrate for the methyltransfer catalyzed by PRMT1 and PRMT5 (H4R3me1).

### 2.2 Antibody Detection of Methylated Product as a Screen Readout

Previously described primary antibody is used to detect the MMA mark. This antibody displays affinity toward H4R3me1, H2AR3me1 and NpmR187me1. In support of the experimental setup, the MMA antibody was used to detect the PRMT5 product NpmR187me1. The experiments are performed under typical nitrocellulose blocking/wash/incubation protocols with an ultrasensitive HRP substrate (TMA-6, Lumigen). Light output was directly quantified with a LAS4000 16-bit digital imager (GE) and had a linear response over a wide range of product concentrations, making this approach a good tool for the screen.

### 2.3 Competitive Assay and Scintillation as a Screen Readout

Previously described filter binding assays (e.g. P81 phosphocellulose) are used to separate radioactive SAM cofactor ([³H-methyl]SAM or [¹⁴C-methyl]SAM) from the radioactive methylated product (e.g. small peptide or full length protein methylated at lysine or arginine residue). At pH=8 (bicarbonate buffer), the methylated radioactive product of the methyltransferase reactions is isolated onto filter binding surface and the radioactivity is further quantified using a scintillation detector.

### 2.4 Profiling of PRMTs Using Antibody Detection

Protocol for PRMT profiling with the SAM analog library. Briefly, buffered 50 μL reactions contain an optimized concentration of PRMT5-MEP50 (or other methyltransferase), sub-saturating levels of acceptor (i.e. concentration equal to Kₘ for the protein substrate) and 25 μM of SAM or SNA probes. Reaction samples are deposited onto nitrocellulose membrane (e.g. 10 μL) and these DotBlots are further analyzed for detection of the MMA mark. In addition to this first reactivity screen, a second experiment is performed using similar conditions where SAM concentration is kept constant (25 μM) and SNA probes are added to compete with the cofactor (final 125 μM concentration). Analysis with the DotBlot approach permits identification of SAM competitive inhibitors. To complete the profiling, Kₘ and k_{cat} are measured for substrate analogs and Kᵢ for inhibitors (SNH or SNC probes). These kinetic parameters are determined under discontinuous conditions through detection of products (Kₘ, k_{cat}) or SAH (Kᵢ) by reverse-phase UPLC.

### 2.5 Profiling of PRMTs Using Filter Assay and Scintillation Counter

Protocol for PRMT Profiling with the SAM Analog Library.

Briefly, buffered 50 μL reactions contain an optimized concentration of PRMT5-MEP50 (or other methyltransferase), sub-saturating levels of acceptor (i.e. concentration equal to Kₘ for the protein substrate), 25 μM of radioactive SAM ([³H-methyl] or [¹⁴C-methyl]) and increasing concentrations of each SNA probes. Reaction samples are deposited onto filter membranes (e.g. P81 phosphocellulose). Membranes are further washed with bicarbonate buffer (pH=8) to remove excess radioactive SAM substrate, leaving the radioactive methylated peptide/protein product onto the filter surface. Dried-out filters are further incubated with scintillation liquid and radioactive signal is determine through scintillation counting. To complete the profiling, Kₘ and k_{cat} are measured for substrate analogs and Kᵢ for inhibitors These kinetic parameters are determined under discontinuous conditions.

### Results

SAM analogs and SAH analogs have been synthesized using the enzymatic approach. These S-Nucleosyl Amino acid probes (SNA) are prepared with excellent yields (>70%) and purified by HPLC. Compounds are characterized using Mass Spectrometry to confirm exact mass.

The SAM analogs are shown below:

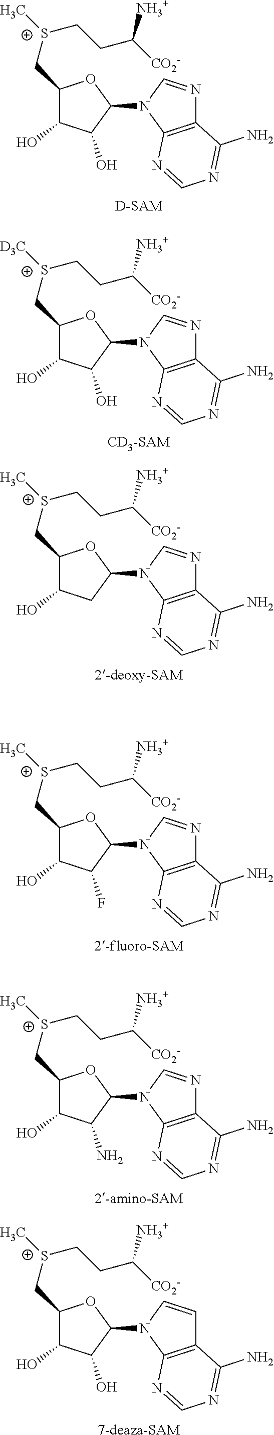

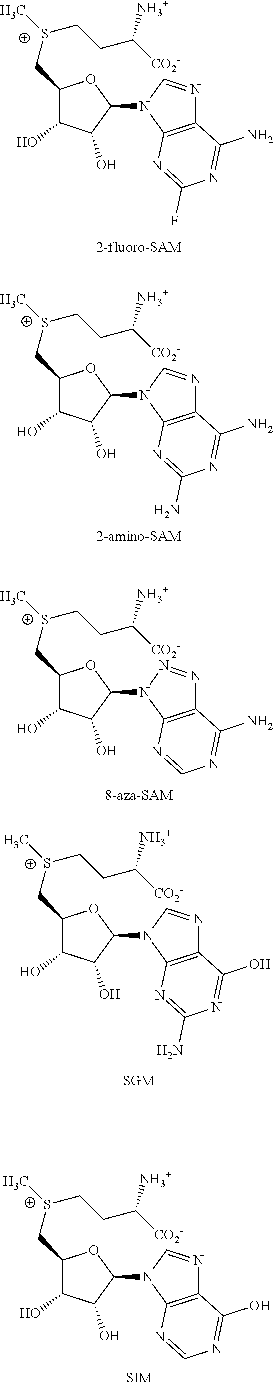

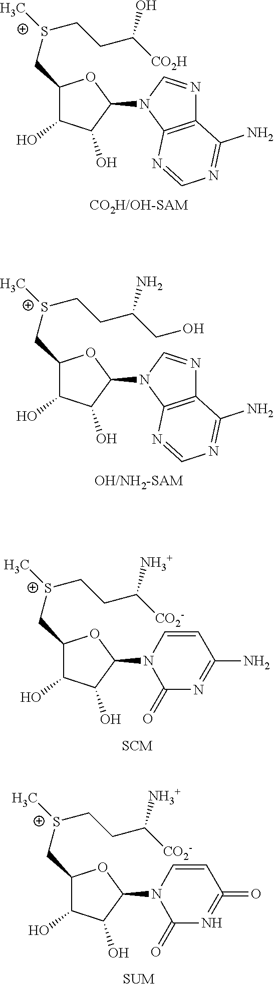

| | m/z | | HPLC (C18 4.6 × 250 mm) | |
|---|---|---|---|---|
| | predicted | experiment | RT (min) | Absₘₐₓ (nm) |
| D-SAM | 399.1445 | 399.1444 | 4.78 | 260 |
| 2′-deoxy-SAM | 383.1496 | 383.1495 | 9.85 | 260 |
| 2′-fluoro-SAM | 401.1402 | 401.1401 | 9.80 | 260 |
| 2′-amino-SAM | 398.1605 | 398.1602 | 3.93 | 260 |
| 7-deaza-SAM | 398.1493 | 398.1496 | 6.56 | 272 |
| 2-fluoro-SAM | 417.1351 | 417.1343 | 5.85 | 262 |
| 2-amino-SAM | 414.1554 | 414.1547 | 4.37 | 257/280 |
| SGM | 415.1394 | 415.1390 | 3.58 | 253 |
| 8-aza-SAM | 400.1398 | 400.1398 | 4.67 | 279 |
| SIM | 400.1285 | 400.1289 | 3.16 | 248 |
| CO₂H/OH-SAM | 400.1285 | 400.1284 | 9.71 | 260 |
| OH/NH₂-SAM | 385.1653 | 385.1652 | 4.03 | 260 |
| SCM | 375.1333 | 375.1332 | 2.77 | 269 |
| SUM | 376.1173 | 376.1172 | 2.85 | 260 |

HPLC Method (Luna2 C18, 4.6×250 mm, 5 μm): Buffer A (Acetic acid and Triethylamine 100 mM, pH 6.0) and Buffer B (same as Buffer A with 30% Acetonitrile). 0-4 min, 1 mL/min 99% A; gradient to 10 min, 1 mL/min 10% A; ramp to 2 mL/min to 10.25 min; constant till 16.25 min with 10% A; maintained at 2 mL/min and return to 99% A to 16.75 min; constant till 22.75 min; ramp down to 0 mL/min at 23 min.

The SAH analogs are shown below:

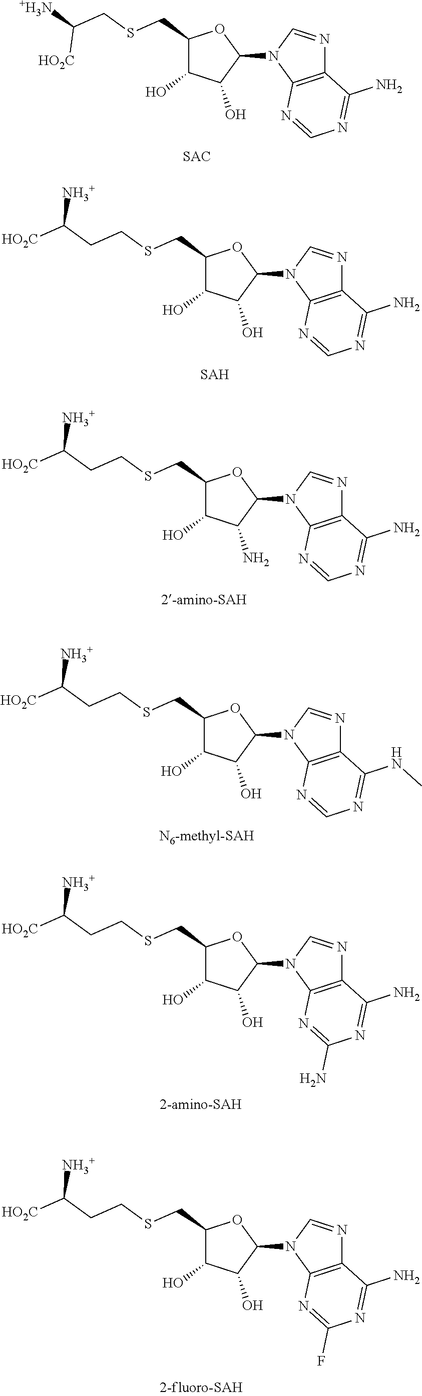 [SMILES: Nc1ncnc2c1ncn2[C@@H]1O[C@H](CSC[C@H]([NH3+])C(=O)O)[C@@H](O)[C@H]1O ; Nc1ncnc2c1ncn2[C@@H]1O[C@H](CSCC[C@H]([NH3+])C(=O)O)[C@@H](O)[C@H]1O ; Nc1ncnc2c1ncn2[C@@H]1O[C@H](CSCC[C@H]([NH3+])C(=O)O)[C@@H](O)[C@H]1N ; CNc1ncnc2c1ncn2[C@@H]1O[C@H](CSCC[C@H]([NH3+])C(=O)O)[C@@H](O)[C@H]1O ; Nc1nc(N)c2ncn([C@@H]3O[C@H](CSCC[C@H]([NH3+])C(=O)O)[C@@H](O)[C@H]3O)c2n1 ; Nc1nc(F)nc2c1ncn2[C@@H]1O[C@H](CSCC[C@H]([NH3+])C(=O)O)[C@@H](O)[C@H]1O]

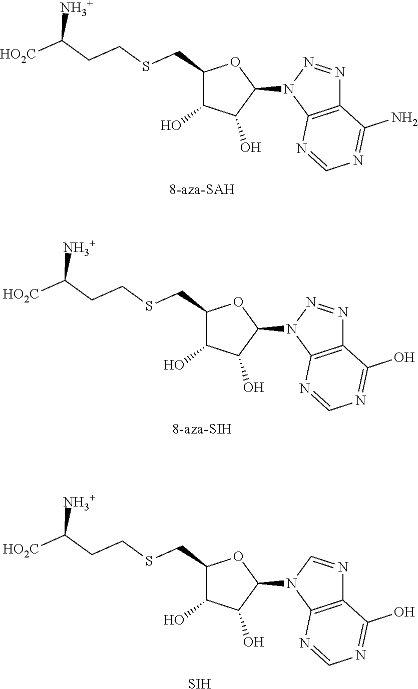 [SMILES: Nc1ncnc2c1nnn2[C@@H]1O[C@H](CSCC[C@H]([NH3+])C(=O)O)[C@@H](O)[C@H]1O ; [NH3+][C@@H](CCSC[C@H]1O[C@@H](n2nnc3c(O)ncnc32)[C@H](O)[C@@H]1O)C(=O)O ; [NH3+][C@@H](CCSC[C@H]1O[C@@H](n2cnc3c(O)ncnc32)[C@H](O)[C@@H]1O)C(=O)O ; "SIH"]

| | m/z | | HPLC (C18 4.6 × 250 mm) | |
|---|---|---|---|---|
| | predicted | experiment | RT (min) | Absₘₐₓ (nm) |
| SAC | 371.1132 | 371.1132 | 10.08 | 260 |
| 2′-amino-SAH | 384.1448 | 384.1447 | 10.19 | 260 |
| N₆-methyl-SAH | 399.1445 | 399.1445 | 10.83 | 260 |
| 2-amino-SAH | 400.1398 | 400.1397 | 10.16 | 257/280 |
| 2-fluoro-SAH | 403.1194 | 403.1195 | 10.63 | 262 |
| 8-aza-SAH | 386.1241 | 386.1242 | 10.24 | 278 |
| 8-aza-SIH | 387.1081 | 387.1081 | 11.60 | 254 |
| SIH | — | — | — | — |

HPLC Method (Luna2 C18, 4.6×250 mm, 5 μm): Buffer A (Acetic acid and Triethylamine 100 mM, pH 6.0) and Buffer B (same as Buffer A with 30% Acetonitrile). 0-4 min, 1 mL/min 99% A; gradient to 10 min, 1 mL/min 10% A; ramp to 2 mL/min to 10.25 min; constant till 16.25 min with 10% A; maintained at 2 mL/min and return to 99% A to 16.75 min; constant till 22.75 min; ramp down to 0 mL/min at 23 min.

### Discussion

Disclosed herein is an approach to rapidly produce a combinatorial collection of S-Nucleosyl Amino acid probes (SNA) that can serve as preferred methyl transferase substrates or inhibitors toward methyltransferases. Specifically, a fast enzymatic synthesis of a compound library of SNA probes can be produced from commercial building blocks using unique biocatalysts. The chemical probes from the initial library display a single point variation compared to the natural SAM/SAH and convolutions with 3 or more point variation can lead to a potential 10⁶ molecule library. This approach is useful to obtain inhibitors with improved isozyme specificity. Since many of these enzymes are oncogenic or otherwise involved in human disease, such targeted compounds will enhance personalized medicine and reduce potential side effects due to off-target inhibition.

The present approach is highly innovative because the platform permits facile screening of a large chemical library to determine SAM cofactor binding specificity for methyl transferases. Small molecules targeting the cofactor binding pocket of these enzymes are likely to yield isozyme-specific inhibitors. Tedious multi-step chemical synthesis of a small number of SAM/SAH analogs was performed in the 1970s and assayed toward four SMMTs. However, these had poor yields (10-30%) with no control over their stereochemistry, showing that a synthetic chemistry approach will be difficult to accomplish. In contrast, the present use of a unique set of natural and powerful catalysts is the key to the stereoselective, cleaner and more efficient synthesis of these analogs.

1. A method of synthesizing an analog of S-adenosyl-L-methionine (SAM) comprising
i) reacting a nucleoside or nucleoside analog with a nucleoside triphosphate in the presence of one or more of adenosine kinase, deoxynucleoside kinase and deoxycytidine kinase to form a monophosphate nucleoside analog;
ii) forming a triphosphate nucleoside analog by
a) reacting the monophosphate nucleoside analog with a nucleoside triphosphate in the presence of one or both of myokinase and cytidylate kinase to form a diphosphate nucleoside analog; and reacting the diphosphate nucleoside analog with a nucleoside triphosphate in the presence of one or both of pyruvate kinase and nucleoside-diphosphate kinase to form a triphosphate nucleoside analog; and/or
b) reacting the monophosphate nucleoside analog with phosphoenolpyruvic acid and pyrophosphate in the presence of pyrophosphate phosphate dikinase to form a triphosphate nucleoside analog; and
iii) reacting the triphosphate nucleoside analog with methionine or a methionine analog in the presence of methionine adenosyltransferase to form an analog of SAM.

2. The method of claim 1, wherein any nucleoside triphosphate is adenosine triphosphate (ATP) or cytidine triphosphate (CTP).

3. The method of claim 1, wherein the adenosine kinase is Anopheles gambiae adenosine kinase.

4. The method of claim 1, wherein the deoxynucleoside kinase is Bacillus anthracis deoxynucleoside kinase.

5. The method of claim 1, wherein the deoxycytidine kinase is Homo sapiens deoxycytidine kinase.

6. The method of claim 1, wherein the cytidylate kinase is Coxiella burnetii cytidylate kinase.

7. The method of claim 1, wherein the nucleoside-diphosphate kinase is Coxiella burnetii nucleoside-diphosphate kinase.

8. The method of claim 1, wherein the pyrophosphate phosphate dikinase is from Zea mays or Clostridium symbiosum.

9. The method of claim 1, wherein the methionine adenosyltransferase is from Methanococcus jannaschii, Sulfolobus solfataricus, Neisseria meningitidis or Campylobacter jejuni.

10. A method of synthesizing an analog of S-adenosyl-L-methionine (SAM) comprising reacting a 5′-chlorinated nucleoside analog with methionine or a methionine analog in the presence of a SAM-dependent chlorinase to form an analog of SAM.

11. The method of claim 10, wherein the SAM-dependent chlorinase is Salinispora tropica enzyme.

12. A method of synthesizing an analog of S-adenosyl-L-homocysteine (SAH) comprising reacting a nucleoside or nucleoside analog with homocysteine or an analog of homocysteine in the presence of S-adenosyl-L-homocysteine hydrolase.

13. (canceled)

14. The method of claim 12, wherein the S-adenosyl-L-homocysteine hydrolase is from Lupinus luteus.

15. The method of claim 1, wherein the nucleoside or nucleoside analog is a ribonucleoside or ribonucleoside analog.

16. The method of claim 1, wherein the nucleoside or nucleoside analog is a 2′-deoxyribonucleoside or 2′-deoxyribonucleoside analog.

17. The method of claim 1, wherein the nucleoside or nucleoside analog is a 3′-deoxyribonucleoside or 3′-deoxyribonucleoside analog.

18. The method of claim 1, wherein the nucleoside or nucleoside analog is adenosine, 2′-deoxyadenosine, 3′-deoxyadenosine, an analog of adenosine, an analog of 2′-deoxyadenosine, or an analog of 3′-deoxyadenosine.

19. (canceled)

20. The method of claim 1, wherein the nucleoside analog is substituted with one or more of methyl, ethyl, benzyl, —NH₂, —OH, —SH, —NHNH₂, —NHOH, —NO₂, —N₃, —F, —Cl, ═O and —CD₃.

21. The method of claim 1, wherein the nucleoside analog is tubercidin, vidarabine, 2-amino adenosine, 2-fluoro adenosine, 2′-fluoro-2′-deoxy adenosine, 2′-amino-2′-deoxy adenosine or N₆-methyl adenosine.

22. The method of claim 1, wherein one or more of adenosine kinase, deoxynucleoside kinase, deoxycytidine kinase, myokinase, cytidylate kinase, pyruvate kinase, nucleoside-diphosphate kinase, pyrophosphate phosphate dikinase, methionine adenosyltransferase, SAM-dependent chlorinase and S-adenosyl-L-homocysteine hydrolase is a recombinant enzyme expressed in E. coli.

23. The method of claim 1, wherein the SAM analog is selected from the group consisting of

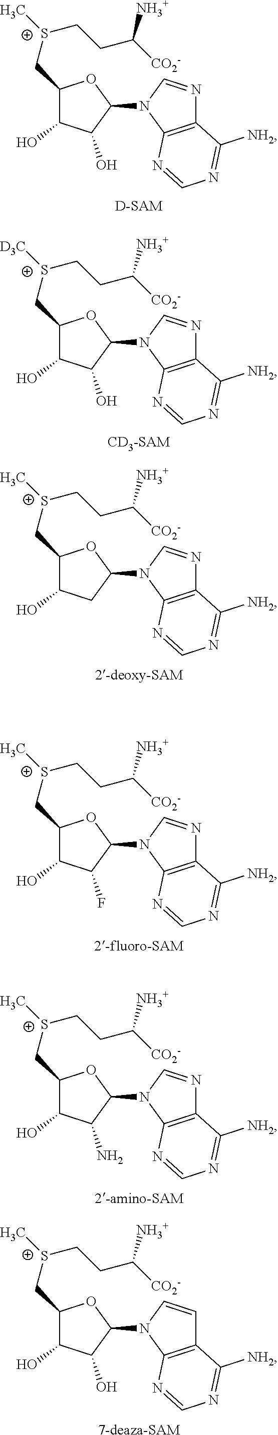

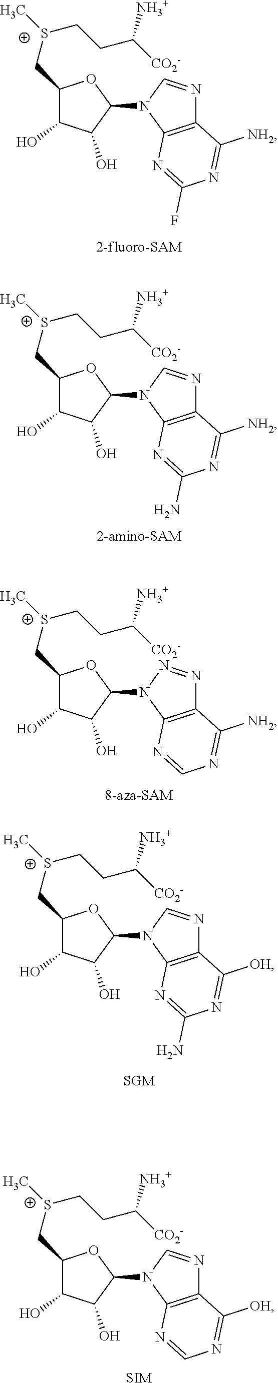

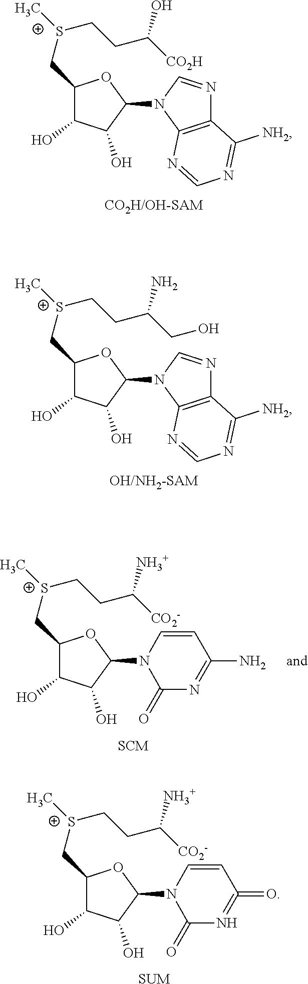

24. The method of claim 12, wherein the SAH analog is selected from the group consisting of

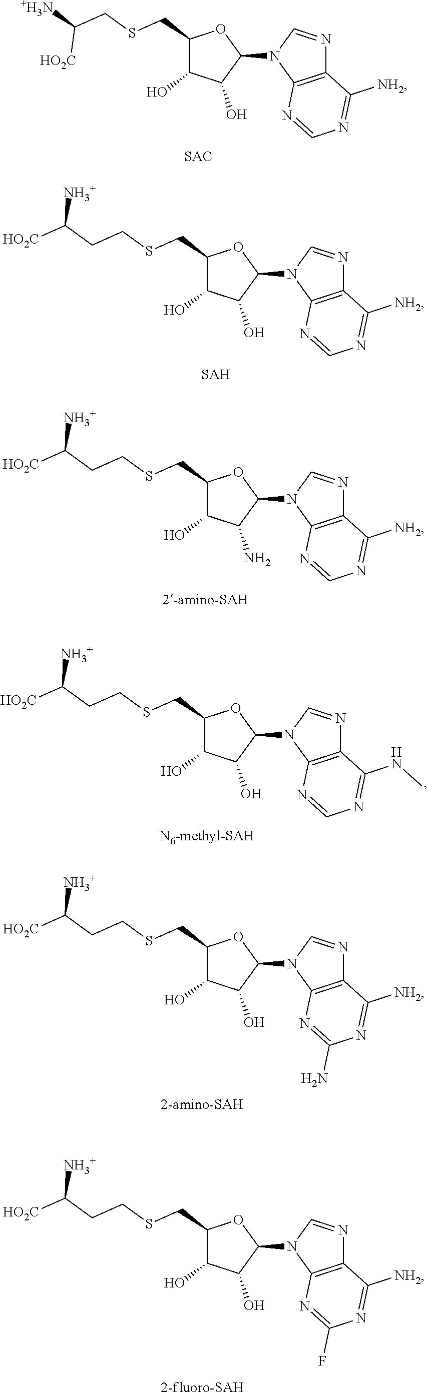 [SMILES: Nc1ncnc2c1ncn2[C@@H]1O[C@H](CSC[C@H]([NH3+])C(=O)O)[C@@H](O)[C@H]1O ; Nc1ncnc2c1ncn2[C@@H]1O[C@H](CSCC[C@H]([NH3+])C(=O)O)[C@@H](O)[C@H]1O ; Nc1ncnc2c1ncn2[C@@H]1O[C@H](CSCC[C@H]([NH3+])C(=O)O)[C@@H](O)[C@H]1N ; CNc1ncnc2c1ncn2[C@@H]1O[C@H](CSCC[C@H]([NH3+])C(=O)O)[C@@H](O)[C@H]1O ; Nc1nc(N)c2ncn([C@@H]3O[C@H](CSCC[C@H]([NH3+])C(=O)O)[C@@H](O)[C@H]3O)c2n1 ; Nc1nc(F)nc2c1ncn2[C@@H]1O[C@H](CSCC[C@H]([NH3+])C(=O)O)[C@@H](O)[C@H]1O]

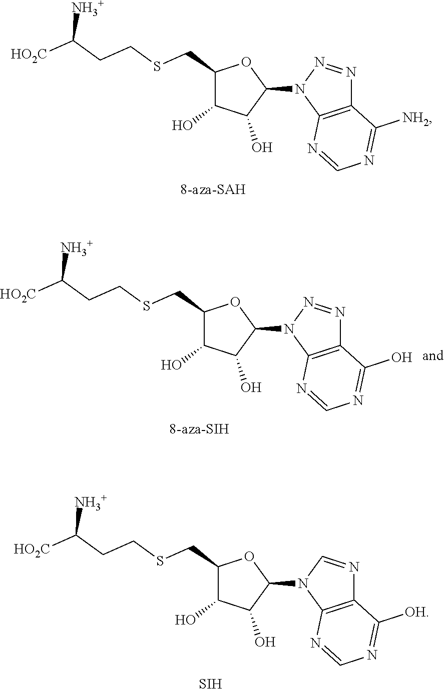 [SMILES: Nc1ncnc2c1nnn2[C@@H]1O[C@H](CSCC[C@H]([NH3+])C(=O)O)[C@@H](O)[C@H]1O ; [NH3+][C@@H](CCSC[C@H]1O[C@@H](n2nnc3c(O)ncnc32)[C@H](O)[C@@H]1O)C(=O)O ; [NH3+][C@@H](CCSC[C@H]1O[C@@H](n2cnc3c(O)ncnc32)[C@H](O)[C@@H]1O)C(=O)O ; "SIH"]

25. A compound selected from the group consisting of 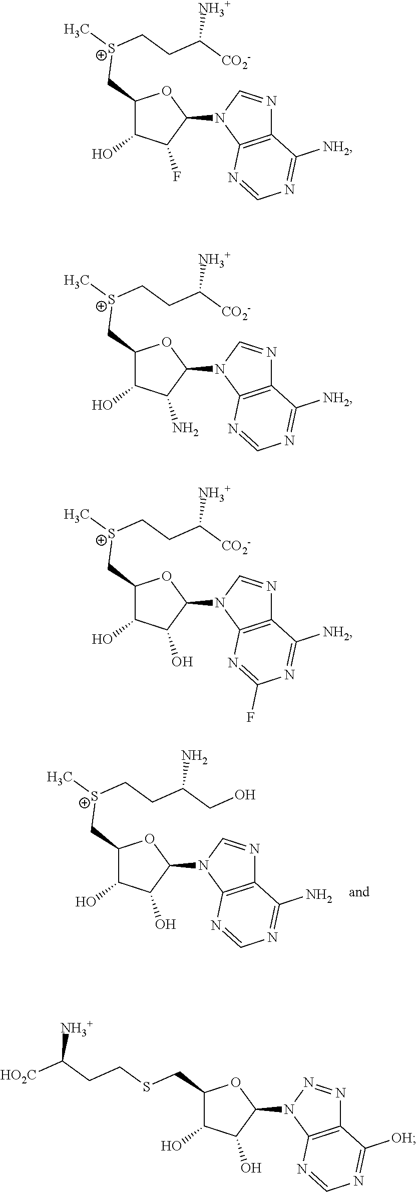
or a pharmaceutically acceptable salt thereof.

26. A pharmaceutical composition comprising one or more of the compounds of claim 25, and a pharmaceutically acceptable carrier.

27. A method of treating a cancer in a subject comprising administering to the subject one or more of the compounds of claim 25 in an amount effective to treat a cancer in a subject.

28. (canceled)
